(19) United States
(12) Patent Application Publication
(10) Pub. No.: US 2022/0175996 A1
(43) Pub. Date: Jun. 9, 2022

(54) AUTOCLAVABLE HUMAN INTERFACE DEVICE

(71) Applicant: Hewlett-Packard Development Company, L.P., Spring, TX (US)

(72) Inventors: Adolfo Gomez, Fort Collins, CO (US); Aaron M. Laaveg, Fort Collins, CO (US); Peter Seiler, Fort Collins, CO (US); Paul Howard Mazurkiewicz, Fort Collins, CO (US)

(73) Assignee: Hewlett-Packard Development Company, L.P., Spring, TX (US)

(21) Appl. No.: 17/599,597

(22) PCT Filed: Jul. 19, 2019
(86) PCT No.: PCT/US2019/042644

§ 371 Date: Sep. 29, 2021

(51) Int. Cl. A61L 2/24; G06F 1/18; C09D 163/00; A61L 2/26; A61L 2/07; B05D 5/00; B05D 7/24

(52) U.S. Cl. CPC A61L 2/24; G06F 1/182; C09D 163/00; G06F 3/0202; A61L 2/07; B05D 5/00; B05D 7/24; A61L 2/26

(57) ABSTRACT

An autoclavable human interface device is described. The autoclavable human interface device can include a first component constructed of a first material exposable to autoclave conditions without damage caused to the first component. The autoclavable human interface device can include a second component constructed of a second material that is susceptible to damage under the autoclave conditions. The autoclavable human interface device can include a component coating on the second component. The component coating can be exposable to the autoclave conditions without allowing damage to the second component.

### BACKGROUND

An autoclave is a pressure chamber used to sterilize an object such as equipment or supplies placed within the autoclave by applying heat, humidity or moisture and/or pressure for a duration of time inside the autoclave. For example, the autoclave can remove or inactivate bacteria, viruses, fungi and/or spores on the object by applying a pressurized saturated steam at a defined temperature and for a defined duration inside the autoclave.

### BRIEF DESCRIPTION OF THE DRAWINGS

FIG. 1 illustrates an example of a system that supports autoclaving in accordance with the present disclosure;

FIG. 2 illustrates an example of a computing system that includes a compute device and an autoclavable human interface device in accordance with the present disclosure;

FIG. 3 is a flowchart illustrating an example method of making an autoclavable human interface device in accordance with the present disclosure;

### DETAILED DESCRIPTION

The present disclosure describes a device as well as a system and a method. An example of the present disclosure can include an autoclavable human interface device. The autoclavable human interface device can include a first component constructed of a first material exposable to autoclave conditions without damage caused to the first component. The autoclavable human interface device can include a second component constructed of a second material that is susceptible to damage under the autoclave conditions. The autoclavable human interface device can include a component coating on the second component. The component coating can be exposable to the autoclave conditions without allowing damage to the second component.

In one example, the first component can be a housing, an electrical component, a key or button, a pigmented ink, a cable, or a combination thereof. In another example, the second component can include an electronics component. In yet another example, the component coating can be in the form of a photo-cured or thermally-cured epoxy resin. In another example, the autoclave conditions can include simultaneous exposure to a temperature from 121° C. to 270° C., and a moisture pressure level from 1 psi to 30 psi above atmospheric pressure for a duration of 10 minutes to 60 minutes. In a further example, the component coating can have a thickness from 1 μm to 5 mm. In yet a further example, the first component and the component coating can be both of polymer construction having a heat deflection temperature from 130° C. to 250° C. In one aspect, the first component can include a perforated surface to facilitate egress of liquid collected within the autoclavable human interface device during autoclaving. In another aspect, the autoclavable human interface device can be detachable from a docking interface.

Another example of the present disclosure can include a computing system. The computing system can include a compute device and an autoclavable human interface device to provide a received input to the compute device. The autoclavable human interface device can include a first component constructed of a first material without damage caused to the first component. The autoclavable human interface device can include a second component constructed of a second material that is susceptible to damage under the autoclave conditions. The autoclavable human interface device can include a component coating on the second component. The component coating can be exposable to the autoclave conditions and can maintain the second component within operational environmental conditions.

In one example, the computing system can include a docking interface to electronically and mechanically receive and permit detachment of the autoclavable human interface device. In another example, the first component can be a housing, an electrical component, a key or button, a pigmented ink, a cable, or a combination thereof. In yet another example, the second component can include an electronics component.

Another example of the present disclosure can include a method of making an autoclavable human interface device. The method can include assembling an autoclavable human interface device that includes a first component and a second component, wherein the first component is constructed of a first material exposable to autoclave conditions without damage caused to the first component, and wherein the second component is constructed of a second material that is susceptible to damage under the autoclave conditions. The method can further include applying a component coating on the second component either before or after assembling the autoclavable human interface device, wherein the component coating on the second component encases the second component at locations susceptible to damage under the autoclave conditions. The component coating can coat the second component to reduce environmental conditions exposed to be within an operational tolerance.

In these examples, it is noted that when discussing the device, the system, or the method, any of such discussions can be considered applicable to the other examples, whether or not they are explicitly discussed in the context of that example. Thus, for example, in discussing details about an autoclavable human interface device, such discussion also refers to the methods and systems described herein, and vice versa.

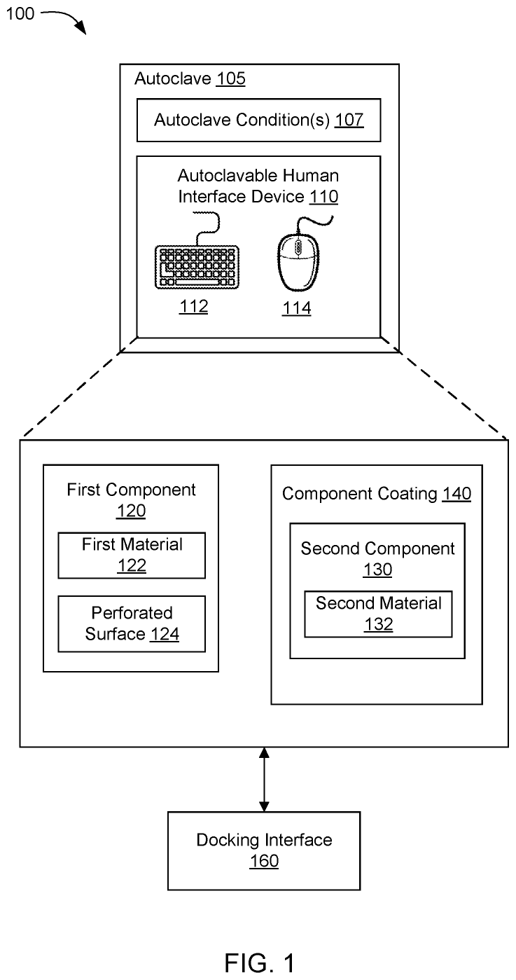

Turning now to the FIGS., FIG. 1 illustrates an example of a system 100 that supports autoclaving. The system 100 can include an autoclave 105 and an autoclavable human interface device 110 that can be placed inside the autoclave 105. The autoclave 105 can apply various autoclave conditions 107 inside the autoclave 105, such as a defined temperature, humidity, moisture and/or pressure for a defined duration of time. The autoclave 105 can apply the autoclave conditions 107 to sterilize the autoclavable human interface device 110 that is placed inside the autoclave 105. For example, the autoclave 105 can apply the autoclave conditions 107 to remove or inactivate bacteria, viruses, fungi and/or spores on the autoclavable human interface device 110.

In one example, the autoclave conditions 107 can involve simultaneous exposure of the autoclavable human interface device 110 to the defined temperature and a moisture pressure level for the defined duration. For example, the defined temperature can be a temperature between 121° C. to 270° C., the moisture pressure level can be between 1 psi and 30 psi above atmospheric pressure, and the duration of time can be between 10 minutes and 60 minutes. The application of the autoclave conditions 107 during an autoclaving process can cause the autoclavable human interface device 110 placed within the autoclave 105 to be sterilized.

In one example, the autoclavable human interface device 110 can be a type of computer device that takes input from humans and/or provides output to humans. An autoclavable human interface device 110 that is capable of receiving an input can include, but is not limited to, a keyboard 112 or a computer mouse 114. In another example, an autoclavable human interface device 110 that is capable of receiving an input can include, but is not limited to, a pointing device such as a trackball, touchpad, pointing stick or light pen, a joystick, a gamepad or game controller, an analog stick, a touchscreen, a magnetic stripe reader, a graphics tablet, a web camera, a microphone or a fingerprint scanner. In addition, an autoclavable human interface device 110 that is capable of producing an output can include, but is not limited to, a computer monitor, a touch display, a refreshable braille display, loudspeakers, ear bud speakers, a headset or haptic technology, which can include head mounted displays that support virtual reality (VR) and/or augmented reality (AR) applications.

In one example, the autoclavable human interface device 110 can include a first component 120 and a second component 130. The first component 120 can be constructed of a first material 122. The first material 122 can be exposable to the autoclave conditions 107 without damage caused to the first component 120. More specifically, the first material 122 can be exposable to the autoclave conditions 107 which include simultaneous exposure to the temperature between 121° C. to 270° C. and the moisture pressure level between 1 psi and 30 psi above atmospheric pressure for the defined duration between 10 minutes and 60 minutes during the autoclaving process without damage caused to the first component 120. Thus, the first component 120 and the second component 130 can be made of materials and construction that are inherently non-susceptible to autoclave-induced damage.

As used herein, “damage” to a component in the autoclavable human interface device 110 such as the first component 120 or the second component 130 can include an autoclave-induced malformation or an autoclave-induced malfunction of the component. As used herein, “damage” to a component may not include superficial or otherwise trivial damages to the component by the autoclave 105.

In one example, the first component 120 constructed of the first material 122 can be, but is not limited to, a housing, an electronics component, a key or button, a pigmented ink, or a cable.

In one example, the housing of the autoclavable human interface device 110 can enclose and protect the various components of the autoclavable human interface device 110. In this example, the first material 122 used to construct the housing can have a polymer construction. For example, the first material 122 can be a type of plastic, including, but not limited to, acrylonitrile butadiene styrene (ABS), polycarbonate ABS (PC/ABS), high impact polystyrene (HIPS), etc. In another example, the first material 122 used to construct the housing can be a material other than plastic, such as aluminum, rubber, silicon, wood, steel, titanium, lead, copper, etc. In this example, the first material 122 used to construct the housing can be exposable to the autoclave conditions 107 without causing damage to the housing.

In one example, the electronics component(s) of the autoclavable human interface device 110 can be used to provide various functionalities of the autoclavable human interface device 110. In this example, the first material 122 used to construct the electronics component(s) can include various metals including, but not limited to, copper, platinum, silver, gold, nickel, cobalt, aluminum, tin, zinc, etc. The electronics component(s) can include active components, passive components and/or electromechanical components. Non-limiting examples of active components can include semiconductors such as diodes, transistors, integrated circuits, or optoelectronic devices, display technologies such as light emitting diode (LED), liquid-crystal display (LCD) or plasma, or power sources. Non-limiting examples of passive components can include resistors, capacitors such as electrolytic capacitors, inductive devices, transducers, sensors, detectors, antennas, etc. Non-limiting examples of electromechanical components include piezoelectric devices, resonators, terminals, connectors, cable assemblies, switches, etc. In this example, the first material 122 used to construct the electronics component(s) can be exposable to the autoclave conditions 107 without causing damage to the electronics component(s).

In one example, the key(s) or button(s) of the autoclavable human interface device 110 can be pressed or interacted with by a user to provide an input to the autoclavable human interface device 110. In this example, the first material 122 used to construct the key(s) or button(s) can have a polymer construction. For example, the first material 122 can be a type of plastic, including, but not limited to, ABS, PC/ABS. HIPS, etc. In this example, the first material 122 used to construct the key(s) or button(s) can be exposable to the autoclave conditions 107 without causing damage to the key(s) or button(s).

In one example, the pigmented ink of the autoclavable human interface device 110 can be used to define the key or button of the autoclavable human interface device 110. For example, the pigmented ink can be used to indicate key letters, numbers and symbols (e.g., A, 1, !), key names, etc. In this example, the first material 122 used to form the pigmented inks can be based on epoxy resins, polyurethanes or polyacrylates. In this example, the first material 122 used to form the pigmented ink can be exposable to the autoclave conditions 107 without causing damage to the pigmented ink.

In one example, the cable of the autoclavable human interface device 110 can connect the autoclavable human interface device 110 to a computing device. For example, the autoclavable human interface device 110 can include the cable when the autoclavable human interface device 110 is not a wireless device. In this example, the first material 122 used to construct the cable can be formed from high temperature grade materials, such as high temperature polyvinyl chloride (PVC), high temperature polyolefin, or high temperature thermoplastic polyurethane (TPU), etc. Other non-limiting examples of the first material 122 used to construct the cable can be ethylene proplyene diene monomer (EPDM), silicone rubber with K-fiber jacket (SRK), polytetrafluoroethylene (PTFE), fluorinated ethylene propylene (FEP), perfluoroalkoxy (PFA), tetrafluoroethylene (TEE), ethylene tetrafluoroethylene (ZW), or styrene-butadiene-systems (SBS) type polymers. In this example, the first material 122 used to form the cable can be exposable to the autoclave conditions 107 without causing damage to the cable.

In one example, the first component 120 constructed of the first material 122 can be a lens for a laser, for example, when the autoclavable human interface device 110 is a computer mouse. In this example, the first material 122 used to construct the lens can include a polymer such as plastic, or glass. The lens can be susceptible to becoming cloudy or becoming damaged when cleaned, in which case light may not be received or transmitted sufficiently to continue operation. Thus, the first material 122 used to form the lens can be exposable to the autoclave conditions 107 without causing damage to the lens.

In one example, the second component 130 can be constructed of a second material 132. The second material 132 may not be exposable to the autoclave conditions 107 without damage caused to the second component 130. In other words, when the second material 132 is exposed to the temperature and/or the moisture pressure level for the defined duration during the autoclaving process, damage can be caused to the second component 130. In this example, the second component 130 by itself can be unable to tolerate the temperature and/or the moisture pressure level for the defined duration during the autoclaving process, and as a result, damage can be caused to the second component 130 during the autoclaving process.

In one example, the autoclavable human interface device 110 can include a component coating 140 on the second component 130. The component coating 140 can be on the second component 130 at locations susceptible to damage under the autoclave conditions 107. The component coating 140 can be exposable to the autoclave conditions 107 without allowing damage to the second component 130 where the component coating 140 is present. In other words, the component coating 140 can fully or partially encase the second component 130 to protect the second component 130 against the autoclave conditions 107. More specifically, the component coating 140 can fully or partially encase the second component 130 to protect the second component 130 against the temperature and the moisture pressure level for the defined duration during the autoclaving process, thereby preventing damage from being caused to the second component 130.

In one example, the component coating 140 can be exposable to the autoclave conditions 107 and can maintain the second component 130 within operational conditions. As used herein, “operational conditions” can refer to the ability of the second component 130 to be fully functional and operational after exposure to the autoclave conditions 107. For example, the component coating 140 can maintain an operation of the second component 130 when exposed to environmental conditions of the autoclave 105. In another example, the component coating 140 can coat the second component 130 to reduce environmental conditions exposed to be within an operational tolerance. For example, the second component 130 may be able to tolerate the autoclave conditions 107 and maintain operation when coated or encased by the component coating 140. As used herein, “operational tolerance” can refer to a tolerance level of the second component 130 to be fully functional and operational after exposure to the autoclave conditions 107. For example, the component coating 140 can cause the second component 130 to be operational under the autoclave conditions 107 until a specific tolerance level is reached or exceeded.

In one example, the second component 130 can be an electronics component or another component that is otherwise vulnerable to temperature, moisture and/or pressure during the autoclaving process. For example, the second component 130 can be a component that is unable to otherwise withstand the temperature and the moisture pressure level for the defined duration during the autoclaving process without the aid of the component coating 140.

In one example, the component coating 140 can be an epoxy or other film-like material, where the component coating 140 can be in the form of a photo-cured or thermally-cured epoxy resin. In another example, the component coating 140 can include, but is not limited to, coatings in the families of epoxy resins, phenolic resins, polyurethanes, polyacrylates (acrylics), silicones, styrene-butadiene systems (SBS), amorphous fluorinated polymers (e.g., PTFE, etc.), polyolefins and aramids.

In one configuration, the first component 120 and the component coating 140 can both be of a polymer construction having a defined heat deflection temperature, a defined grass transition temperature and/or a defined softening temperature that allows the first component 120 and the component coating 140 to withstand the autoclave conditions 107 without causing damage to the first component 120 or the second component 130 that is fully or partially encased within the component coating 140. The heat deflection temperature (or heat distortion temperature) can indicate a temperature at which a polymer or plastic deforms under a specified load. The glass transition temperature (Tg) can indicate a temperature range over which a glass transition occurs, where the glass transition refers to the gradual and reversible transition in amorphous materials (such as polymers) from a hard and relatively brittle state into a viscous or rubbery state as a temperature is increased. The softening temperature can indicate a temperature at which a material softens beyond an arbitrary level of softness. In one example, the first component 120 and the component coating 140 can have a defined heat deflection temperature, a defined grass transition temperature and/or a defined softening temperature that is above a threshold or is above a threshold by a certain margin, which allows the first component 120 and the component coating 140 to withstand the autoclave conditions 107.

As a non-limiting example, the first component 120 and the component coating 140 can have a heat deflection temperature and/or a glass transition temperature between 130° C. to 250° C., thereby enabling the first component 120 and the component coating 140 to withstand the autoclave conditions 107.

In one example, the component coating 140 can have a defined thickness, which enables the component coating 140 to protect the second component 130 from the autoclave conditions 107. As a non-limiting example, the component coating 140 can have a thickness between 1 μm to 5 mm, which can allow the second component 130 to be protected from the autoclave conditions 107.

In one configuration, the first component 120 (e.g., a housing) can incorporate or employ design elements that serve to prevent accumulation of liquids and vapor during the autoclaving process. For example, the design elements can facilitate egress of liquid or vapor collected within the autoclavable human interface device 110 during the autoclaving process through assisted techniques, such as centrifugal drying. The design elements can also assist in the drying or removal process after the autoclavable human interface device 110 has been processed through the autoclave 105. In a specific example, the first component 120 may include a perforated surface 124, which can allow for drainage of remnant liquid or vapor accumulated during the autoclaving process.

In one configuration, the system 100 can include a docking interface 160. The docking interface 160 can electronically and mechanically receive and permit detachment of the autoclavable human interface device 110. For example, the autoclavable human interface device 110 can attach to the docking interface 160 for charging or when the autoclavable human interface device 110 is not being used. The docking interface 160 may be unable to withstand the autoclave conditions 107. When the autoclavable human interface device 110 is desired to be used and/or sterilized using the autoclave 105, the autoclavable human interface device 110 can be detached from the docking interface 160.

In one configuration, human interface device surfaces, such as keyboard and computer mouse surfaces, are routinely subjected to microbial exposure. Certain environments, such as a medical laboratory environment, can inherently be at increased risk of microbial exposure, and thus contamination of human interface device surfaces. In certain environments and/or under certain conditions, the risk and/or consequences of the transmission of microbes can be severe, such as in hospital settings where a sub-set of the population can be immune-compromised, or in public spaces where microbial transmission pathways can be difficult to control. The microbial exposure pathways can include transfer from user contact, deposition of environmental contaminants and/or transfer from other equipment.

In one example, human interface device surfaces could be sanitized by spraying a chemical solution and wiping the human interface device surfaces clean. In another example, human interface devices could employ removable covers that are washable and/or wipeable. However, using chemical solvents to clean human interface devices may not be fully effective at killing or inactivating microbes, and the chemical solvents could include substances that potentially cause damage to electronic and mechanical components of the human interface devices over time.

In one example, autoclaves can be used in medial environments to sterilize equipment. Autoclaves can be used to sterilize equipment that has an ability to withstand increased temperatures, humidities, and pressures. As a non-limiting example, autoclaves can operate at approximately 121° C., using pressurized saturated steam, and exposure times of up to 20 minutes to sterilize the equipment. However, typical human interface devices could not be compatible with autoclaves. For example, autoclaves could present a risk of electromechanical damage to a typical human interface device, which can include heat deformation of polymeric materials used in both functional and cosmetic parts of the typical human interface device, as well as the melting of solder used in the manufacturing and assembly of electrical components in the typical human interface device.

In the present disclosure, an autoclavable human interface device is described that is capable of withstanding an autoclaving process performed by an autoclave, without damage to the various components included in the autoclavable human interface device. The autoclavable human interface device can include components that are constructed using construction materials selected to have glass transition temperatures or melting points that withstand the energy of a typical autoclave operation. These construction materials can include metals having melting temperatures greater than a temperature applied in an autoclave and/or high-performance polymers having glass transition temperatures greater than a temperature applied in an autoclave. By selecting construction materials that have a suitable melting temperature or glass transition temperature, the autoclavable human interface device can be constructed to withstand repeated autoclaving sessions in an autoclaving environment.

Further, in the present disclosure, the autoclavable human interface device can include printed and flexible circuit board assemblies that use solders with melting temperatures greater than a temperature applied in an autoclave, thereby ensuring nominal electrical operation after an autoclaving process. In addition, the autoclavable human interface device can include electrical components capable of withstanding increased moisture and humidity, as well as resisting corrosion, when exposed to liquid and vapor in an increased pressure and temperature autoclaving environment.

In one example, in the present disclosure, the autoclavable human interface device can employ pigmented inks to indicate keys, features, functions, etc., and the pigmented inks can be selected to withstand the autoclaving environment. More specifically, pigmented inks that do not smear or run from a surface under typical autoclave conditions can be selected for use in the autoclavable human interface device.

In the present disclosure, construction materials including metals and polymers, circuit board assemblies including solder, pigmented inks, etc. that are capable of withstanding an autoclaving environment without damage or deformity can be selected when designing and manufacturing an autoclavable human interface device. Further, the construction materials, circuit board assemblies, pigmented inks, etc. can be selected based on the melting temperature, glass transition temperature, or other metrics that indicate the capability of exposure to heat, moisture and pressure in an autoclaving environment without incurring damage.

In one configuration, in the present disclosure, certain electrical components can be protected from the autoclaving environment by encasing, either partially or fully, the electrical component into an epoxy or similar type of coating that when cured can withstand the moisture and pressure applied in the autoclaving environment. The epoxy or other coating can serve to seal and insulate the electrical component from the autoclave environment. Thus, certain electrical components that would otherwise not be able to withstand the autoclave environment can be encased or covered with the epoxy or coating, thereby protecting the electrical components from exposure to the moisture and pressure in the autoclaving environment without damage caused to the electrical components.

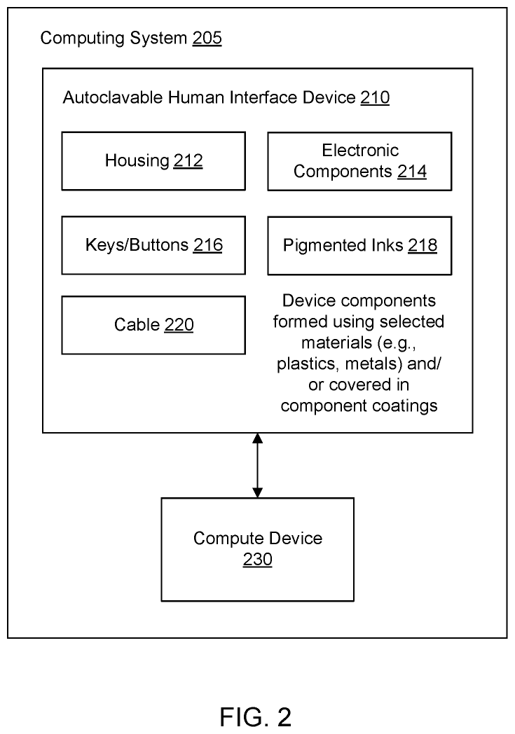

FIG. 2 illustrates an example of a computing system 205 that includes a compute device 230 and an autoclavable human interface device 210. The compute device 230 can be a processor-based device, which can include but is not limited to, a desktop computer, laptops or notebook computers, tablet computers, mobile devices, mainframe computer systems, desktop workstations, mobile workstations, server, network computers, televisions, gaming systems, etc. The autoclavable human interface device 210 can include, but is not limited to, a keyboard, a computer mouse, a game controller, a joystick, a remote control, etc. The autoclavable human interface device 210 can be capable of withstanding repeated autoclaving procedures. In other words, the autoclavable human interface device 210 and components within the autoclavable human interface device 210 can be capable of withstanding autoclave conditions which include simultaneous exposure to a defined temperature and a defined moisture pressure level for a duration of time without damage caused to the autoclavable human interface device 210 and its components.

In one example, the autoclavable human interface device 210 can include various device components, such as a housing 212, electrical or electronics components 214, keys or buttons 216, pigmented inks 218 and/or a cable 220. The device components can be formed using selected construction materials (e.g., plastics, metals) and/or encased in component coatings that offer protection during autoclaving. For example, the housing 212 and the keys/buttons 216 can be constructed using materials (e.g., polymers) having a glass transition temperature and/or a heat deflection temperature that satisfies a defined criteria. For example, the glass transition temperature and/or a heat deflection temperature can be above a defined threshold or above a defined threshold by a certain margin. As a result, the housing 212 and the keys/buttons 216 may be able to withstand exposure to the heat, moisture, etc. during the autoclaving process without damage caused to the housing 212 and the keys/buttons 216. Further, the electronic components 214 can be constructed using materials (e.g., metals) having a melting temperature that satisfies a defined criteria, which can result in the electronic components 214 being able to withstand the autoclaving process without damage to the electronic components 214. In some cases, the electronic components 214 can be encased in component coatings to protect against moisture and/or humidity applied during the autoclaving process. Further, the pigmented inks 218 and the cable 220 can be formed using selected materials having properties that satisfy a defined criteria. Thus, the pigmented inks 218 and the cable 220 may be able to withstand exposure to the heat, moisture, etc. during the autoclaving process without damage caused to the pigmented inks 218 and the cable 220.

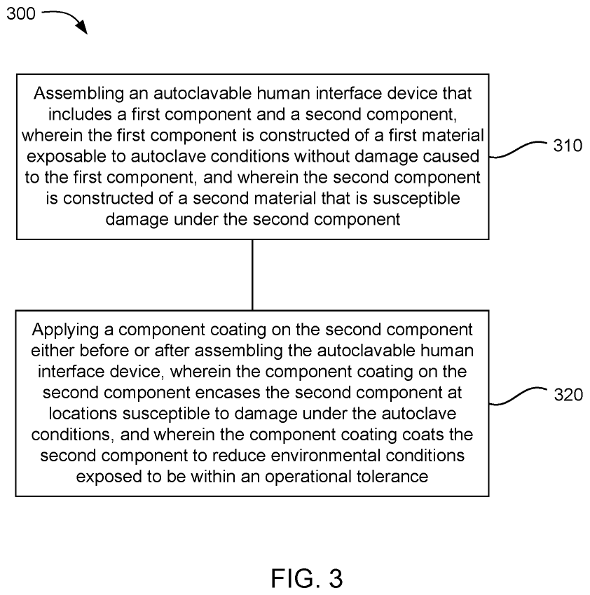

FIG. 3 is a flowchart illustrating one example method 300 of making an autoclavable human interface device. The method can include assembling an autoclavable human interface device that includes a first component and a second component, wherein the first component is constructed of a first material exposable to autoclave conditions without damage caused to the first component. The autoclave conditions can include simultaneous exposure to a temperature from 121° C. to 270° C., and a moisture pressure level from 1 psi to 30 psi above atmospheric pressure for a duration of 10 minutes to 60 minutes. The second component can be constructed of a second material that is susceptible to damage under the autoclave conditions, as in block 310. The method can include applying a component coating on the second component either before or after assembling the autoclavable human interface device, wherein the component coating on the second component encases the second component at locations susceptible to damage under the autoclave conditions, and wherein the component coating is exposable to the autoclave conditions without allowing damage to the second component, as in block 320.

While the flowcharts presented for this disclosure can imply a specific order of execution, the order of execution can differ from what is illustrated. For example, the order of two more blocks can be rearranged relative to the order shown. Further, multiple blocks shown in succession can be executed in parallel or with partial parallelization. In some configurations, block(s) shown in the flow chart can be omitted or skipped. A number of counters, state variables, warning semaphores, or messages can be added to the logical flow for purposes of enhanced utility, accounting, performance, measurement, troubleshooting or for similar reasons.

Reference was made to the examples illustrated in the drawings, and specific language was used herein to describe the same. It will nevertheless be understood that no limitation of the scope of the disclosure is thereby intended. Alterations and further modifications of the features illustrated herein, and additional applications of the examples as illustrated herein, are to be considered within the scope of the description.

Furthermore, the described features, structures, or characteristics can be combined in a suitable manner. In the preceding description, numerous specific details were provided, such as examples of various configurations to provide a thorough understanding of examples of the described disclosure. The disclosure may be practiced without some of the specific details, or with other methods, components, devices, etc. In other instances, some structures or operations are not shown or described in detail to avoid obscuring aspects of the disclosure.

Although the subject matter has been described in language specific to structural features and/or operations, it is to be understood that the subject matter defined in the appended claims is not limited to the specific features and operations described above. Rather, the specific features and acts described above are disclosed as example forms of implementing the claims. Numerous modifications and alternative arrangements can be devised without departing from the scope of the described disclosure.

What is claimed is:

1. An autoclavable human interface device, comprising:
a first component constructed of a first material exposable to autoclave conditions without damage caused to the first component;
a second component constructed of a second material that is susceptible to damage under the autoclave conditions; and
a component coating on the second component, wherein the component coating is exposable to the autoclave conditions without allowing damage to the second component.

2. The autoclavable human interface device of claim 1, wherein the first component is a housing, an electrical component, a key or button, a pigmented ink, a cable, or a combination thereof.

3. The autoclavable human interface device of claim 1, wherein the second component includes an electronics component.

4. The autoclavable human interface device of claim 1, wherein the component coating is in the form of a photo-cured or thermally-cured epoxy resin.

5. The autoclavable human interface device of claim 1, wherein the autoclave conditions include simultaneous exposure to a temperature from 121° C. to 270° C., and a moisture pressure level from 1 psi to 30 psi above atmospheric pressure for a duration of 10 minutes to 60 minutes.

6. The autoclavable human interface device of claim 1, wherein the component coating has a thickness from 1 μm to 5 mm.

7. The autoclavable human interface device of claim 1, wherein the first component and the component coating are both of polymer construction having a heat deflection temperature from 130° C. to 260° C.

8. The autoclavable human interface device of claim 1, wherein the first component includes a perforated surface to facilitate egress of liquid collected within the autoclavable human interface device during autoclaving.

9. The autoclavable human interface device of claim 1, wherein the autoclavable human interface device is detachable from a docking interface.

10. A computing system, comprising:
a compute device; and
an autoclavable human interface device to provide a received input to the compute device, wherein the autoclavable human interface device includes:
a first component constructed of a first material exposable to autoclave conditions without damage caused to the first component,
a second component constructed of a second material that is susceptible to damage under the autoclave conditions, and
a component coating on the second component, wherein the component coating is exposable to the autoclave conditions and maintains the second component within operational environmental conditions.

11. The computing system of claim 10, further comprising a docking interface to electronically and mechanically receive and permit detachment of the autoclavable human interface device.

12. The computing system of claim 10, wherein the first component is a housing, an electrical component, a key or button, a pigmented ink, a cable, or a combination thereof.

13. The computing system of claim 10, wherein the second component includes an electronics component.

14. A method of making an autoclavable human interface device, comprising:
assembling an autoclavable human interface device that includes a first component and a second component, wherein the first component is constructed of a first material without damage caused to the first component, and wherein the second component is constructed of a second material that is susceptible to damage under the autoclave conditions; and
applying a component coating on the second component either before or after assembling the autoclavable human interface device, wherein the component coating on the second component encases the second component at locations susceptible to damage under the autoclave conditions, and wherein the component coating coats the second component to reduce environmental conditions exposed to be within an operational tolerance.

15. The method of claim 14, wherein the first component and the component coating are both of polymer construction having a heat deflection temperature from 130° C. to 260° C.
